(19) United States
(12) Patent Application Publication
(10) Pub. No.: US 2023/0040357 A1
(43) Pub. Date: Feb. 9, 2023

(54) PHARMACEUTICAL COMPOSITION AND HEALTH FUNCTIONAL FOOD FOR PREVENTING, AMELIORATING OR TREATING DIABETES CONTAINING WASP EXTRACT AS ACTIVE INGREDIENT

(71) Applicant: Andong National University Industry-Academic cooperation Foundation, Gyeongsangbuk-do (KR)

(72) Inventor: Ho Yong SOHN, Daegu (KR)

(21) Appl. No.: 17/553,503

(22) Filed: Dec. 16, 2021

(30) Foreign Application Priority Data
Jul. 27, 2021 (KR) 10-2021-0098686

(51) Int. Cl. A61K 35/64; A23L 29/00

(52) U.S. Cl. CPC A61K 35/64; A23L 29/03

(57) ABSTRACT

An antibiotic composition containing a wasp extract as an active ingredient, and more particularly, a pharmaceutical composition and a health functional food for preventing, ameliorating or treating diabetes by α-amylase inhibition and α-glucosidase inhibition, which contain a wasp extract as an active ingredient. The wasp extract exhibits strong antidiabetic activity by effectively inhibiting amylase, has no human erythrocyte hemolytic activity, has excellent heat stability, and the activity thereof is not lost even under an acidic condition of pH 2 and in plasma. The wasp extract of the present disclosure may be processed into various forms such as extracts, powders, pills or tablets, which can be taken at all times.

### BACKGROUND

The present disclosure relates to an antibiotic composition containing a wasp extract as an active ingredient, and more particularly, to a pharmaceutical composition and a health functional food for preventing, ameliorating or treating diabetes by α-amylase inhibition and α-glucosidase inhibition, which contain a wasp extract as an active ingredient.

Diabetes mellitus, which is one of the most common diseases among modern people, is a type of metabolic disease caused by insufficient insulin secretion or insulin secretion dysfunction, is characterized by a high blood glucose level, and causes various symptoms and signs due to hyperglycemia. Diabetes is classified into type 1 and type 2. It is known that type 1 diabetes is caused by the inability to produce insulin at all due to genetic influence, and type 2 diabetes is caused by insulin resistance due to poor insulin function. In particular, it is known that type 2 diabetes is caused mainly by high-calorie, high-fat and high-protein diet due to westernization of diet, lack of exercise, and environmental factors such as stress. For the treatment of diabetes, insulin injection is essential for the treatment of type 1 diabetes, and lifestyle modification and drug treatment are required for the treatment of type 2 diabetes. Typically, insulin secretagogues (e.g., repaglinide, mitiglinide, etc.) and agents [Glucoby, ingredient name: acarbose; Basin, ingredient name: voglibose] for delaying carbohydrate absorption in the small intestine, etc. are used for the treatment of diabetes.

Meanwhile, wasps are insects belonging to the family Vespidae of the order Hymenopterathe, and are predatory insects that eat bees and other small insects. There are about 30 wasp species of 5 genera (Vespa, Vespula, Dolichovespula, Parapolybia, and Polistes) worldwide. In Korea, the following 10 wasp species are known: Vespa analis parallela Andre, V. baslis Smith, V. ducalis Smith, V. dybowskii Andre, Vespa crabro crabroniformis Smith, V. crabro flavofasciata Cameron, V. mandarinia Smith, V. simillima Smith, V. smillima xanthoptera Cameron, and V. velutina nigrithorax. Wasps have different habitats and distributions depending on the species, build wasp nests and inhabit in groups mainly in low mountains, treetops or in the ground.

Wasps are also responsible for pollination and perform the roles of parasites and predators. In particular, some wasps are recognized as pests in the beekeeping industry because they prey on adult honeybees, and damage by them is gradually increasing. In recent years, wasps have been a threat to humans and animals due to wasp stings, and multiple stings with the powerful wasp venom can sometimes cause death. Thus, in order to prevent damage caused by wasps, studies on wasp attraction and capture (Jung Jun-Seong et al., 2018. Trends in Agriculture & Life Sciences 56: 35-45) and the control of wasps using a specific frequency (Kim et al., 2019. J. Apiculture 34: 7-13) have been conducted.

In Korea, wasps have been recognized as a nourishing tonic food since ancient times, and as a representative example, wasp wine made by soaking wasps in alcohol has been used for the treatment of inflammation, epilepsy, convulsions and dental diseases (Heo Jun's Donguibogam, 1615). However, studies on wasps are very limited, and most of the existing studies are studies on ecology and classification of wasps (Murat et al., 2016, Carbohydrate Polymers 145: 64-70) and wasp venom (Xinwang et al., 2013, Toxicon 74: 151-157: Yoon et al., 2015, Journal of Asia-Pacific Entomology 18: 815-823), and there are no studies on the useful physiological activity of wasps themselves.

A study on wasp venom has reported the strong hemolytic activity and cytotoxicity of the wasp venom, and recently, anti-inflammatory activity, allergy induction and neurotoxicity by histamine, serotonin, phospholipase A2, hyaluronidase, etc. have been found (Lee, B. H., Park, H. J. 1998. J. Korean Environ. Sci. Sco. 62-66; Sabe et al., 2017. Med. Inflamm. 6978194. doi: 10.1155/2017/6978194). However, until now, there has been no report on the antidiabetic activity of a wasp extract.

Patents related to wasps include Korean Patent No. 10-2028363 [entitled “System for combating harmful wasps”] and Korean Patent No. 10-2075057 [entitled “Wasp trapping device capable of collecting wasps alive”], Korean Patent No. 10-1972070 [entitled “Composition for preventing or treating gout containing bee venom isolated from worker bees of V. mandarinia Smith”], Korean Patent No. 10-1972074 [entitled “Composition for preventing or treating Alzheimer's disease containing bee venom isolated from worker bees of V. mandarinia Smith”], Korean Patent No. 10-1374327 [entitled “Functional cosmetic composition containing V. mandarinia Smith venom extract”], Korean Patent Application Publication No. 10-1999-0039050 [entitled “Agent for preventing and treating acne containing wasp venom extract”], Korean Patent Application Publication No. 10-2012-0111206 [entitled “Method of preparing functional cosmetic composition using wasps”], Korean Patent Application Publication No. 10-2012-0100450 [entitled “Method of preparing functional cosmetic composition using V. mandarinia Smith”], and Korean Patent Application Publication No. 10-2005-0028992 [“Method of producing traditional wine using wasps and traditional wine liquor using wasps produced thereby”]. However, until now, there has been no known patent related to the antidiabetic activity of a wasp extract.

Prior Art Documents: 1) KR 10-1972070; 2) KR 10-2012-0100450 A.

### SUMMARY OF THE INVENTION

The present disclosure has been made in order to solve the above-described problems occurring in the prior art, and an object of the present disclosure is to provide a pharmaceutical composition and health functional food having antidiabetic activity, which contain a wasp extract as an active ingredient.

To achieve the above object, the present disclosure provides a pharmaceutical composition for preventing or treating diabetes containing a wasp extract as an active ingredient.

The wasp is preferably selected from the group consisting of Vespa mandarinia Smith, Vespa simillima simillima Smith, and Vespa velutina nigrithorax.

The extract is preferably an ethanol extract.

The present disclosure also provides a health functional food for preventing or ameliorating diabetes containing a wasp extract as an active ingredient.

The wasp is preferably selected from the group consisting of Vespa mandarinia Smith, Vespa simillima simillima Smith, and Vespa velutina nigrithorax.

The extract is preferably an ethanol extract.

### BRIEF DESCRIPTION OF THE DRAWINGS

FIGURE shows photographs of V. mandarinia Smith, V. simillima simillima Smith and Vespa velutina nigrithorax used in the Examples of the present disclosure.

### DETAILED DESCRIPTION OF THE INVENTION

Hereinafter, the present disclosure will be described in detail.

In order to test the antidiabetic efficacy of wasps, the present inventors first collected V. mandarinia Smith, V. simillima simillima Smith and Vespa velutina nigrithorax in Korea, and then washed the collected wasps with water to remove foreign substances, followed by soaking in 30% ethanol and 1 year of leaching. Thereafter, the extracts were filtered and concentrated under reduced pressure, and the extracts were recovered as an antidiabetic active ingredient. It was confirmed that the active ingredient is characterized by having excellent heat stability and acid stability. Thus, the active ingredient was intended to be used as an active ingredient for a pharmaceutical composition and a health functional food for preventing, ameliorating or treating diabetes.

Specifically, in order to develop a pharmaceutical composition and a health functional food for preventing, ameliorating or treating diabetes by using a wasp extract known to be effective in treating inflammation, epilepsy, convulsions and dental diseases, the present inventors prepared 30% ethanol extracts of wasps and evaluated the inhibitory activities of these extracts against α-amylase and α-glucosidase. As a result, it was confirmed that all the V. mandarinia Smith extract, the V. simillima simillima Smith extract and the Vespa velutina nigrithorax extract exhibits antidiabetic activity by a certain level of α-glucosidase inhibition. In particular, it was confirmed that the V. simillima simillima Smith extract exhibits the best α-glucosidase inhibitory activity.

Therefore, the present disclosure provides a pharmaceutical composition for preventing or treating diabetes containing a wasp extract as an active ingredient.

The wasp is preferably selected from the group consisting of Vespa mandarinia Smith, Vespa simillima simillima Smith, and Vespa velutina nigrithorax.

The extract is preferably an ethanol extract.

The present disclosure also provides a health functional food for preventing or ameliorating diabetes containing a wasp extract as an active ingredient.

The wasp is preferably selected from the group consisting of Vespa mandarinia Smith, Vespa simillima simillima Smith, and Vespa velutina nigrithorax.

The extract is preferably an ethanol extract.

Hereinafter, a method for preparing the wasp extract of the present disclosure and a test for the efficacy of the wasp extract will be described in more detail.

In order to achieve the object of the present disclosure, the present inventors performed an experimental method including steps of: collecting three wasp species; preparing wasp extracts; evaluating the antidiabetic activities of the extracts; and examining the stability of the extracts.

The wasp extract contained in the composition of the present disclosure may be obtained by a method including steps of: extracting wasps with 30% ethanol for about 1 year; and filtering the extract through a filter having a mesh size of 0.06 mm or less and concentrating the filtrate under reduced pressure.

A solvent that is used in the present disclosure may be water (cold or hot water), spirit, an anhydrous or hydrous lower alcohol having 1 to 4 carbon atoms (e.g., methanol, ethanol, spirit, propanol, butanol, etc.), or a mixed solvent of the lower alcohol and water, and hot-water extraction or ethanol extraction is most preferred.

In a preferred embodiment of the present disclosure, wasps may be extracted with ethanol. In addition, the ethanol extract may be fractionated sequentially with organic solvents (hexene, ethyl acetate and butanol) or fractioned with each of the organic solvents to additionally obtain a hexene fraction, an ethyl acetate fraction, a butanol fraction and water residue.

The wasp extract of the present disclosure may be prepared into powder through a conventional powdering process such as vacuum drying, freeze-drying, or spray-drying. The wasp extract is not degraded by various degrading enzymes in plasma, and remains active even upon heat treatment at 100° C. and in the human stomach at pH 2.

The wasp extract of the present disclosure exhibits strong inhibitory activity on α-glucosidase, and thus may be used as a material for a pharmaceutical composition and a health functional food for preventing, ameliorating or treating type 1 diabetes, type 2 diabetes, or diabetic complications such as diabetic retinopathy and diabetic nephropathy.

In a preferred embodiment, the antidiabetic composition of the present disclosure may be applied as a pharmaceutical composition.

For use, the pharmaceutical composition containing the active ingredient of the present disclosure may be formulated in various forms, including oral dosage forms such as powders, granules, tablets, capsules, suspensions, emulsions, syrups, and aerosols, or injection dosage forms sterile injections, according to conventional methods depending on the intended use thereof, and may be administered through various routes including oral, intravenous, intraperitoneal, subcutaneous, rectal and topical routes.

This pharmaceutical composition may further contain a carrier, excipient or diluent. Examples of a carrier, excipient or diluent that may be contained in the pharmaceutical composition include lactose, dextrose, sucrose, sorbitol, mannitol, xylitol, erythritol, maltitol, starch, gum acacia, alginate, gelatin, calcium phosphate, calcium silicate, cellulose, methyl cellulose, amorphous cellulose, polyvinyl pyrrolidone, water, methylhydroxybenzoate, propylhydroxybenzoate, talc, magnesium stearate, and mineral oil. In addition, the pharmaceutical composition of the present disclosure may further contain a filler, an anti-aggregating agent, a lubricant, a wetting agent, a flavoring agent, an emulsifying agent, a preservative, and the like.

In a preferred embodiment, solid formulations for oral administration include tablets, pills, powders, granules, capsules, and the like, and such solid formulations are prepared by mixing the pharmaceutical composition with at least one excipient, for example, starch, calcium carbonate, sucrose, lactose, gelatin, and the like. In addition to simple excipients, lubricants such as magnesium stearate, talc and the like may also be used.

In a preferred embodiment, liquid formulations for oral administration include, for example, a suspension, an internal solution, an emulsion, a syrup, and the like. In addition to water and liquid paraffin, which are commonly used simple diluents, various excipients, for example, a wetting agent, a sweetening agent, a flavoring agent, a preservative, and the like may be contained.

In a preferred embodiment, formulations for parenteral administration include, for example, sterile aqueous solutions, non-aqueous solutions, suspensions, emulsions, lyophilized formulations, suppositories, and the like. As non-aqueous solvents or suspending agents, there may be used propylene glycol, polyethylene glycol, vegetable oil such as olive oil, injectable ester such as ethyl oleate, and the like. Injections may contain conventional additives such as a solubilizing agent, an isotonic agent, a suspending agent, an emulsifying agent, a stabilizing agent, a preservative, and the like.

The active ingredient of the present disclosure is administered in a pharmaceutically effective amount. As used herein, the term “pharmaceutically effective amount” refers to an amount sufficient to treat a disease at a reasonable benefit/risk ratio applicable to any medical treatment. The effective dose level of the active ingredient may be determined depending on factors, including the kind and severity of the patient's disease, the activity of the drug, sensitivity to the drug, the time of administration, the route of administration, excretion rate, the duration of treatment, and drugs used in combination with the composition, as well as other factors well known in the medical field. The pharmaceutical composition of the present disclosure may be administered individually or in combination with other therapeutic agents, and may be administered sequentially or simultaneously with conventional therapeutic agents. The pharmaceutical composition may be administered in a single or multiple dosage form. It is important to administer the pharmaceutical composition in the minimum amount that can exhibit the maximum effect without causing side effects, in view of all the above-described factors, and this amount can be easily determined by a person skilled in the art.

In a preferred embodiment, the effective amount of the active ingredient in the pharmaceutical composition of the present disclosure may vary depending on the patient's age, sex and body weight. Generally, the active ingredient may be administered daily or every other day at a dose of 1 to 5,000 mg/kg body weight, preferably 100 to 3,000 mg/kg body weight, or may be administered 1 to 3 times a day at this dose. However, since the dose may increase or decrease depending on the route of administration, the severity of the disease, the patient's sex, weight and age, etc., the dose is not intended to limit the scope of the present disclosure in any way.

The pharmaceutical composition of the present disclosure may be administered to a subject through various routes. All modes of administration can be contemplated, and for example, the composition may be administered orally, intrarectally, or by intrarectal, intravenous, intramuscular, subcutaneous, intrauterine, intrathecal or intracerebroventricular injection.

In the present disclosure, the term “administration” means providing a given substance to a patient by any suitable method. The pharmaceutical composition of the present disclosure may be administered orally or parenterally through all general routes as long as it can reach the target tissue. In addition, the composition of the present disclosure may also be administered using any device capable of delivering the active ingredient to target cells.

In the present disclosure, the term “subject” is not particularly limited, but includes, for example, humans, monkeys, cattle, horses, sheep, pigs, chicken, turkeys, quails, cats, dogs, mice, rats, rabbits or guinea pigs, and preferably refers to mammals, more preferably humans.

In addition, the health functional food of the present disclosure may be variously used in foods and beverages that are effective in preventing and ameliorating diabetes. Foods containing the active ingredient of the present disclosure include various foods, for example, beverages, gums, teas, vitamin complexes, health supplement foods and the like, and may be used in the form of powders, granules, tablets, capsules or beverages.

The active ingredient of the present disclosure may generally be added in an amount of 0.01 to 15 wt % based on the total food weight. For a health beverage composition, the active ingredient may be added in an amount of 0.02 to 10 g, preferably 0.3 to 1 g, based on 100 ml of the health beverage composition.

The health functional food of the present disclosure may additionally contain food-acceptable additives, for example, natural carbohydrates and various flavoring agents, in addition to containing the wasp extract as an essential component at the indicated percentage.

Examples of the natural carbohydrates include conventional sugars, such as monosaccharides (e.g., glucose, fructose, etc.), disaccharides (e.g., maltose, sucrose, etc.), polysaccharides (e.g., dextrin, cyclodextrin, etc.), and sugar alcohols such as xylitol, sorbitol, erythritol or the like.

Examples of the flavoring agents that may be used in the present disclosure include thaumatin, rebaudioside A, glycyrrhizin, saccharin, aspartame, etc. The flavoring agent is used in an amount of about 1 to 20 g, preferably about 5 to 12 g, based on 100 mL of the health functional food of the present disclosure. In addition, the health functional food of the present disclosure may contain various nutrients, vitamins, minerals, flavoring agents such as synthetic flavoring agents and natural flavoring agents, colorants, extenders, pectic acid and its salt, alginic acid and its salt, organic acids, protective colloidal thickeners, pH adjusting agents, stabilizers, preservatives, glycerin, alcohol, carbonizing agents that are used in carbonated beverages, etc. Additionally, the health functional food of the present disclosure may contain fruit flesh that is used for the preparation of natural fruit juice, fruit juice beverages or vegetable beverages. These components may be used individually or in combination. The content of these additives is generally selected in the range of 0.01 to about 20 parts by weight based on 100 parts by weight of the health functional food.

Hereinafter, the present disclosure will be described in more detail with reference to specific examples. The following examples merely describe one preferred embodiment of the present disclosure, and the scope of the present disclosure is not limited by the contents described in the following examples.

### Example 1: Wasp Collection and Wasp Extract Preparation

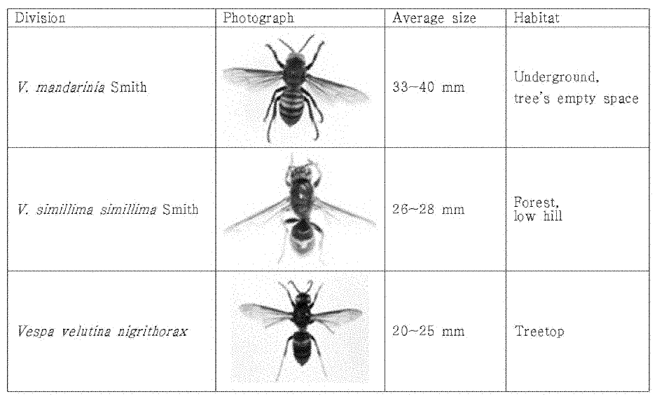

In August 2019, V. mandarinia Smith, V. simillima simillima Smith and Vespa velutina nigrithorax were collected in Gyeongsangbuk-do, Korea, and each wasp species was identified by an insect expert, Andong University professor Jeong Cheol-Hee (FIG. 1). The collected wasps were soaked in 30% ethanol without separate pretreatment and extracted therein for about 1 year. In this case, about 5 liters of 30% ethanol was added to 100 wasps. Thereafter, the wasps were removed, and the wasp extracts were filtered, and dried under reduced pressure and recovered.

### Example 2: Analysis of Components of Wasp Extracts

The total polyphenol, total flavonoid, total sugar and reducing sugar contents in each of the wasp extracts prepared in Example 1 were measured. For measurement of the total polyphenol content, 50 μl of Folin-ciocalteu and 100 μl of Na₂CO₃ saturated solution were added to 400 μl of each extract sample and allowed to stand at room temperature for 1 hour, and then the absorbance at 725 nm was measured. As a standard reagent, tannic acid was used. For measurement of the total flavonoid content, each extract sample was extracted by stirring with methanol for 18 hours and filtered, and 4 ml of 90% diethylene glycol was added to each of the filtrate samples and 40 μl of 1N NaOH was added thereto, and each of the mixtures was allowed to react at 37° C. for 1 hour, and then the absorbance at 420 nm was measured. As a standard reagent, rutin was used. Reducing sugar was quantified using a DNS method, and total sugar was quantified using a phenol-sulfuric acid method.

TABLE 1
Analysis of components of wasp extracts

| | Content (mg/g) | | | |
|---|---|---|---|---|
| Division | Total polyphenol | Total flavonoid | Total sugar | Reducing sugar |
| V. mandarinia Smith | 33.5 ± 0.9 | 3.5 ± 0.1 | 83.0 ± 6.3 | 54.8 ± 0.0 |
| Vespa velutina | 40.1 ± 0.1 | 8.0 ± 2.5 | 115.2 ± 4.8 | 60.6 ± 11.5 |
| nigrithorax | | | | |
| V. simillima simillima | 29.8 ± 3.8 | 4.2 ± 0.4 | 121.2 ± 3.3 | 53.6 ± 1.6 |
| Smith | | | | |

As a result, as shown in Table 1 above, the extracts of the three wasp species showed a total polyphenol content of 29.8 to 40.1 mg/g, and the Vespa velutina nigrithorax extract showed the highest total polyphenol content. The total flavonoid content was 3.5 to 8.0 mg/g and was the highest in the Vespa velutina nigrithorax extract. However, the total sugar content was the highest in the V. simillima simillima Smith extract (121.1 mg/g), which was 1.46 times higher than that in the V. mandarinia Smith extract, and the reducing sugar content was 53.6 to 60.6 mg/g and did not significantly differ between the extracts.

### Example 3: Evaluation of Antidiabetic Activities of Wasp Extracts

The antidiabetic activities of the wasp extracts prepared in Example 1 were evaluated. Specifically, the in-vitro α-amylase inhibitory activity and α-glucosidase inhibitory activity of each extract were evaluated.

First, for evaluation of the α-amylase inhibitory activity, 2.5 μl of each extract sample was mixed with 25 μl of α-amylase (0.25 U/ml) diluted with 50 mM phosphate buffer (pH 6.8). The mixture was allowed to react at 37° C. for 10 minutes, and then allowed to react with 25 μl of 0.5% soluble starch (Samchun Chemicals Co., Korea) at 37° C. for 10 minutes and then heated at 100° C. for 5 minutes, followed by stopping of the reaction. 150 μl of DNS (3,5-dinitrosalicylic acid, Sigma Co., St. Louis, USA) was added to the reaction solution which was then color-developed by heating at 100° C. for 5 minutes and allowed to cool at room temperature. The color-developed solution was measured for its absorbance at 540 nm, and the inhibition (%) of α-amylase was calculated.

Inhibition (%)=[1−(enzymatic activity of sample-added group/enzymatic activity of control-added group)]×100

The α-glucosidase inhibitory activity was evaluated using pNPG (p-nitrophenol glucoside; Sigma Co., USA). Specifically, 2.5 μl of a wasp extract sample was mixed with 25 μl of α-glucosidase (0.25 U/ml) diluted with 50 mM sodium acetate buffer (pH 5.6), and the mixture was allowed to react at 37° C. for 10 minutes, and then allowed to react with 25 μl of 1 mM pNPG solution at 60° C. for 10 minutes. Thereafter, the reaction was stopped by adding 25 μl of 1 M NaOH. The absorbance at 405 nm was measured and the α-glucosidase inhibition (%) was calculated.

Inhibition (%)=[1−(enzymatic activity of sample-added group/enzymatic activity of control-added group)]×100

TABLE 2
Antidiabetic activities of wasp extracts

| | Antidiabetic activity | |
|---|---|---|
| | α-amylase | α-glucosidase |
| Division | inhibition (%) | inhibition (%) |
| V. mandarinia Smith | −3.9 ± 0.6 | 8.3 ± 2.2 |
| Vespa velutina nigrithorax | −5.8 ± 3.9 | 10.0 ± 1.5 |
| V. simillima simillima Smith | 4.6 ± 0.0 | 13.4 ± 0.5 |
| Acarbose (0.5) | 47.6 ± 1.3 | 68.1 ± 0.1 |
| Acarbose (0.063) | 39.8 ± 1.2 | 35.2 ± 5.3 |

As a result, as shown in Table 2, acarbose, which is used as a therapeutic agent for diabetes in clinical practice, showed α-amylase and α-glucosidase inhibitory activities in a concentration-dependent manner, confirming the evidence for using acarbose clinical use. It was confirmed that the extracts of the three wasp species all exhibited excellent α-glucosidase inhibitory activity, and in particular, the V. simillima simillima Smith extract exhibited the strongest α-glucosidase inhibitory activity. The V. simillima simillima Smith extract also exhibited α-amylase inhibitory activity, unlike the V. mandarinia Smith and Vespa velutina nigrithorax extracts. Thus, it was confirmed that the wasp extracts, particularly the V. simillima simillima Smith extract, can be used for the prevention and treatment of type 2 diabetes.

### Example 4: Evaluation of Erythrocyte Hemolytic Activities of Wasp Extracts

In order to evaluate the acute toxicities of the wasp extracts, the human erythrocyte hemolytic activities of the wasp extracts were evaluated, and the results of the evaluation are shown in Table 3 below. At this time, the hemolytic activity was evaluated according to a previous report (Son Ho-Yong et al., 2014, Korean J. Microbiol. Biotechnol. 42: 285-292). Briefly, 100 μl of human erythrocytes were washed three times with PBS and added to a 96-well microplate, and 100 μl of a sample solution at various concentrations was added thereto and then allowed to react at 37° C. for 30 minutes. Thereafter, the reaction solution was centrifuged at 1,500 rpm for 10 minutes, and 100 μl of the supernatant was transferred to a fresh microtiter plate, and then the degree of hemoglobin leakage following hemolysis was measured at 414 nm. DMSO (2%) was used as a solvent control for the sample, and Triton X-100 (1 mg/ml) was used as an experimental control for hemolysis of erythrocytes. Hemolytic activity was calculated using the following equation.

(%) Hemolysis=[(Abs. S−Abs. C)/(Abs. T−Abs. C)]×100

Abs. S: absorbance of sample-added group

Abs. C: absorbance of DMSO-added group

Abs. T: absorbance of Triton X-100-added group

TABLE 3
Human erythrocyte hemolytic activities of wasp extracts

| | Concentration | Human erythrocyte |
|---|---|---|
| Sample/control | (mg/ml) | hemolytic activity |
| Distilled water | — | 0.0 ± 0.3 |
| Solvent (DMSO) | — | 1.5 ± 1.0 |
| Triton X-100 | 1.0 | 100.0 ± 0.1 |
| Amphotericin B | 0.1 | 95.8 ± 0.3 |
| | 0.05 | 72.4 ± 5.2 |
| | 0.025 | 59.5 ± 2 |
| | 0.0125 | 48.2 ± 5.9 |
| | 0.0063 | 21.6 ± 3.7 |
| | 0.0032 | 6.54 ± 0.9 |
| | 0 | 0.0 ± 1.7 |
| V. mandarinia Smith | 1.0 | 11.2 ± 0.2 |
| Vespa velutina nigrithorax | 1.0 | 7.1 ± 0.5 |
| V. simillima simillima Smith | 1.0 | 0.7 ± 0.7 |

As shown in Table 3, it was confirmed that DMSO and water used as controls had no hemolytic activity, and Triton X-100 hemolyzed 100% of erythrocytes at a concentration of 1 mg/ml. Amphotericin B, which is used as an anticancer agent, hemolyzed 59% or more of erythrocytes at a concentration of 0.025 mg/ml. Meanwhile, the wasp extracts showed no erythrocyte hemolytic activity even at a concentration of 1.0 mg/ml. Thus, it is expected that the wasp extracts will show no separate acute toxicity.

### Example 5: Evaluation of Plasma, Acid and Heat Stabilities of Wasp Extracts

The heat stabilities and acid stabilities for antidiabetic activities of the wasp extracts obtained in Example 1 were evaluated. The extracts maintained excellent activity without significant loss of their amylase inhibitory activity even when they were heat-treated at 100° C. for 1 hour or treated at pH 2 (0.01 M HCl) for 1 hour. Therefore, it is expected that the wasp extracts will maintain their heat resistance and acid resistance even in various food processing processes.

As demonstrated through the Examples of the present disclosure, the wasp extract as an active ingredient for a pharmaceutical composition and a health functional food for preventing, ameliorating or treating diabetes according to the present disclosure exhibits strong antidiabetic activity by effectively inhibiting amylase, has no human erythrocyte hemolytic activity, has excellent heat stability, and the activity thereof is not lost even under an acidic condition of pH 2 and in plasma. The wasp extract of the present disclosure may be processed into various forms such as extracts, powders, pills or tablets, which can be taken at all times. Thus, the wasp extract is very useful in the pharmaceutical and food industries.

What is claimed is:

1. A pharmaceutical composition for preventing or treating diabetes containing a wasp extract as an active ingredient.

2. The pharmaceutical composition of claim 1, wherein the wasp is selected from the group consisting of Smith Vespa mandarinia Smith, Vespa simillima simillima Smith, and Vespa velutina nigrithorax.

3. The pharmaceutical composition of claim 1, wherein the extract is an ethanol extract.

4. A health functional food for preventing or ameliorating diabetes containing a wasp extract as an active ingredient.

5. The health functional food of claim 4, wherein the wasp is selected from the group consisting of Vespa mandarinia Smith, Vespa simillima simillima Smith, and Vespa velutina nigrithorax.

6. The health functional food of claim 4, wherein the extract is an ethanol extract.
